(19) United States
(12) Patent Application Publication
(10) Pub. No.: US 2011/0183995 A1
(43) Pub. Date: Jul. 28, 2011

(54) ELTOPRAZINE FOR SUPPRESSION OF L-DOPA INDUCED DYSKINESIAS

(75) Inventors: Anders Björklund, Lund (SE); Munolo Carta, Carbonia (IT); Jørgen Buus Lassen, Bagsvaerd (DK)

(73) Assignee: Neurosearch A/S, Ballerup (DK)

(21) Appl. No.: 13/001,058

(22) PCT Filed: Jun. 23, 2009
(86) PCT No.: PCT/EP2009/057773

§ 371 Date: Mar. 18, 2011

Related U.S. Application Data
(60) Provisional application No. 61/075,132, filed on Jun. 24, 2008.

(30) Foreign Application Priority Data
Jun. 24, 2008 (DK) PA 2008 00865

(51) Int. Cl. A61K 31/496; C07D 403/10; A61P 25/16

(52) U.S. Cl. 514/254.11; 544/377

(57) ABSTRACT

This invention relates to the use of the drug Eltoprazine for combating dyskinesias arising from chronic Levodopa (L-DOPA) therapy in patients suffering from Parkinson's disease.

### TECHNICAL FIELD

This invention relates to the use of the drug Eltoprazine for combating dyskinesias arising from chronic Levodopa (L-DOPA) therapy in patients suffering from Parkinson's disease.

### BACKGROUND ART

Parkinson's disease is the second most common neurodegenerative disorder with an incidence of about 2% in the population over 60 years. The disease is characterized by a loss of dopamine neurons in the substantia nigra pars compacta, which in turns determines a decreased release of dopamine in the corpus striatum. The loss of dopamine input into the striatum is largely responsible for the motor symptoms, such as bradykinesia, postural instability, resting tremor and rigidity.

The precursor of dopamine, L-DOPA (3,4-dihydroxyphenylalanine) is highly effective in relieving the motor symptoms during the first years of administration (the so-called “honeymoon period”). However, over time, patients start experiencing side effects, such as development of abnormal involuntary movements (AIM), known as dyskinesias, following administration of the drug. It has been reported that within 5 years about 50% of the patients develop these motor complications in response to L-DOPA administration. This percentage rises to about 90% after the first decade. The appearance of dyskinesias represents therefore a serious limitation to the use of this therapeutic agent in advanced disease. Currently there are not effective drugs to counteract the side effect of L-DOPA and prolog its therapeutic efficacy.

Recently progress has been made in the comprehension of the mechanism behind the appearance of dyskinesias. Importantly, we have recently shown that dysregulated dopamine release from serotonin terminals, after L-DOPA administration, plays a key role in the emergence of this side effect (see Carta et al.: Serotonin Neuron Transplants Exacerbate L-DOPA-Induced Dyskinesias in a Rat Model of Parkinson's Disease; J. Neurosci. 2007 27 (30) 8011-8022; and Carta et al.: Dopamine released from 5-HT terminals is the cause of L-DOPA-induced dyskinesia in parkinsonian rats; Brain 2007 130 (7) 1819-1833). In fact, removal of the serotonin innervation by a toxin lesion, or pharmacological silencing of the release from the serotonin neurons by a combination of 5-HT_{1A} and 5-HT_{1B} autoreceptor agonists, resulted in a complete suppression of L-DOPA-induced dyskinesias in L-DOPA-primed parkinsonian rats.

The 5-HT_{1A} partial agonist Sarizotan has been recently tested for its antidyskinetic properties, not only in MPTP-treated monkeys, but also in PD patients (see Goetz et al.: Sarizotan as a treatment for dyskinesias in Parkinson's disease: a double-blind placebo-controlled trial; Mov. Disord. 2007 22 (2) 179-186; and Olanow et al.: Multicenter, open-label, trial of sarizotan in Parkinson disease patients with levodopa-induced dyskinesias (the SPLENDID Study); Clin. Neuropharmacol. 2004 27 (2) 58-62). However, despite promising results in the pre-clinical studies, a large phase III clinical trial was recently terminated for lack of efficacy. In light of our results, it seems possible that targeting the 5-HT_{1A} receptors alone is not sufficient to provide a significant control of the excessive swings in L-DOPA-derived dopamine release and therefore of dyskinesias. According to our rodent and primate data, simultaneous activation of the 5-HT_{1A} and 5-HT_{1B} receptors results in a potent synergistic effect and in a more complete control of the motor side effects of L-DOPA medication.

Furthermore, as 5HT_{1A} agonists have shown neuroprotective efficacy in clinically-relevant experimental designs in animal models of PD, including MPTP-treated macaques (see Bezard et al.: A call for clinically driven experimental design in assessing neuroprotection in experimental Parkinsonism; Behav. Pharmacol. 2006 17 (5-6) 379-382), an early introduction of such drug could potentially slow down the neurodegenerative process, further supporting the case of this class of drugs.

Eltoprazine (i.e. 1-(2,3-Dihydro-benzo[1,4]dioxin-5-yl)piperazine) is a 5-HT_{1A} and 5-HT_{1B} receptor agonist, known for its antiagressive properties (see Sijbesma et al.: The anti-aggressive drug eltoprazine preferentially binds to 5-HTIA and 5-HT receptor subtypes in rat brain: sensitivity to guanine nucleotides; European Journal of Pharmacology 1990 187 209-223). The use of Eltoprazine for suppression of dyskinesias arising from chronic L-DOPA therapy has never been reported. However, our recent results, and the peculiar pharmacological profile which combines a 5-HT_{1B} and 5-HT_{1A} receptor agonist activity make Eltoprazine a most promising compound for the treatment of L-DOPA-induced dyskinesia.

### SUMMARY OF THE INVENTION

Based on experiments in rats we believe that Eltoprazine may be considered the single most efficient drug for suppressing the dyskinesias arising from chronic treatment of patients suffering from Parkinson's disease with L-DOPA, without worsening the therapeutic effect of L-DOPA. The efficacy of Eltoprazine is thought due to its unique pharmacological profile, combining an equal 5-HT_{1A} and 5-HT_{1B} receptor agonist activity. Co-activation of these two types of receptor has shown to generate a potent synergistic effect in the suppression of L-DOPA-induced dyskinesia in rats, and in the absence of any detrimental effect on the therapeutic efficacy of the L-DOPA medication.

An additional advantage of Eltoprazine comes from the fact that a combined 5-HT_{1A} and 5-HT_{1B} agonist treatment, when administered over two week period in drug naïve animals, has been found to induce a long-lasting protection against the development of dyskinesia. Thus, animals chronically treated with the combination of 5-HT_{1A} and 5-HT_{1B} receptor agonists are resistant to dyskinesia even when treatment is switched to L-DOPA alone. The use of Eltoprazine according to the present invention therefore also may be considered a protective means, preventing the development of dyskinesias arising from chronic L-DOPA treatment.

Other objects of the invention will be apparent to the person skilled in the art from the following detailed description and examples.

### DETAILED DISCLOSURE OF THE INVENTION

Eltoprazine (i.e. 1-(2,3-Dihydro-benzo[1,4]dioxin-5-yl)piperazine) is a partial 5-HT_{1A} and 5-HT_{1B} receptor agonist, known for its antiagressive properties. The drug may be represented by Formula I,

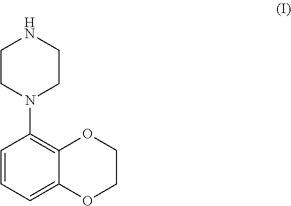

including pharmaceutically acceptable addition salts thereof, and prodrugs thereof.

The present invention relates to the use of Eltoprazine as a medicament for the treatment, prevention or alleviation of dyskinesias arising from the treatment of a subject suffering from Parkinson's disease with L-DOPA (3,4-dihydroxyphenylalanine).

In a preferred embodiment Eltoprazine hydrochloride (i.e. [1-(2,3-dihydro-1,4-benzodioxin-5-yl) piperazine hydrochloride] is employed.

Viewed from another aspect, the invention thus provides a highly potent combination therapy for combating L-DOPA-induced dyskinesias without worsening the therapeutic effect of L-DOPA.

Viewed from a further aspect, the invention provides a medicament intended for co-administration with L-DOPA to a subject suffering from Parkinson's disease, thus providing a combination therapy for combating the abnormal involuntary movements, known as dyskinesias, following administration of the L-DOPA. The medicament may be applied simultaneously with L-DOPA, in a sequential manner, or by separate administration. Preferably Eltoprazine is given at the same time as L-DOPA, or just before, preferably 15-30 minutes before the administration of L-DOPA.

In a preferred embodiment Eltoprazine may be applied (co-administered with L-DOPA) at an early stage of Parkinson's disease, before dyskinesias start appearing in order to prevent or defer the development of dyskinesias. As a preventive therapy, co-administration of Eltoprazine may in particular be accomplished upon initiation of the L-DOPA therapy.

In another preferred embodiment Eltoprazine may be applied (co-administered with L-DOPA) at a later stage of Parkinson's disease, i.e. when dyskinesias start appearing, or have already appeared.

In a third preferred embodiment Eltoprazine may be applied (co-administered with L-DOPA) as intermittent treatment in the therapy, i.e. when wash-out periods may emerge to be necessary to maintain the full therapeutic efficacy of the drug.

It is currently believed that Eltoprazine may be used (co-administered with L-DOPA) in a therapeutically-effective amount in the range of about 0.01-1000 mg API daily, more preferred in the range of about 1-500 mg API daily, even more preferred in the range of about 10-200 mg API daily.

Eltoprazine may be co-administered by any conventional route.

In a preferred embodiment Eltoprazine may be administered either orally, intravenously, intravascularly, intraperitoneally, sub-cutaneously, intramuscularly, inhalatively, topically, by patch, or by suppository.

In a more preferred embodiment Eltoprazine is administered orally (p.o.).

In another more preferred embodiment Eltoprazine is administered intravenously (i.v.).

Any combination of two or more of the embodiments described herein is considered within the scope of the present invention.

### Pharmaceutically Acceptable Salts

The compound (Eltoprazine) for use according to the invention may be provided in any form suitable for the intended administration. Suitable forms include pharmaceutically (i.e. physiologically) acceptable salts, and pre- or prodrug forms of the compound for use according to the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a compound for use according to the invention and its pharmaceutically acceptable acid addition salt.

Examples of pharmaceutically acceptable cationic salts of a compound for use according to the invention include, without limitation, the sodium, the potassium, the calcium, the magnesium, the zinc, the aluminium, the lithium, the choline, the lysine, and the ammonium salt, and the like, of a compound for use according to the invention containing an anionic group. Such cationic salts may be formed by procedures well known and described in the art.

### Prodrugs

The chemical substance for use according to the invention may be administered as such or in the form of a suitable prodrug. The term “prodrug” denotes a compound, which is a drug precursor and which, following administration and absorption, release the drug in vivo via some metabolic process.

Particularly favoured prodrugs are those that increase the bioavailability of the compounds of the invention (e.g. by allowing an orally administrered compound to be more readily absorbed into the blood) or which enhance delivery of the parent compound to a specific biological compartment (e.g. the brain or lymphatic system).

Thus examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are esters or amides.

### Methods of Preparation

The compounds for use according to the invention are well known and commercially available. Alternatively they may readily be prepared by conventional methods from commercially available chemicals.

### Biological Activity

The invention provides a highly potent pharmacological therapy for the treatment of L-DOPA-induced dyskinesia without worsening the therapeutic effect of L-DOPA.

In our experimental conditions, Eltoprazine, at low dose in combination with Buspirone, a 5-HT_{1A} partial agonist, or at higher dose alone was able to produce a near-complete suppression of L-DOPA-induced dyskinesia in rats (see FIG. 2).

Although Eltoprazine has never been tested in non-human primate models of Parkinson's disease, we have recently found that the potent synergistic effect between 5-HT_{1A} and 5-HT_{1B} receptor agonists, previously reported in rats, is also present in the MPTP-treated macaques. Thus, targeting 5-HT_{1A} and 5-HT_{1B} receptors produced a near-complete suppression of dyskinesia also in the monkey Parkinson model.

A further advantage of Eltoprazine that combines a 5-HT_{1A} and 5-HT_{1B} activity, is its capacity to induce long-lasting protection from the development of dyskinesia in animals (rats) treated with the combination of 5-HT_{1A} and 5-HT_{1B} agonists from the very first dose of L-DOPA. Such rats remain protected from a full development of dyskinesia even when agonist treatment is discontinued and animals are given L-DOPA alone (see FIG. 3)

### Pharmaceutical Compositions

In another aspect the invention provides novel pharmaceutical compositions comprising a therapeutically effective amount of the compound for use according to the invention.

While a compound for use according to the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the compound for use according to the invention, or a pharmaceutically acceptable salt or derivative thereof, together with one or more pharmaceutically acceptable carriers, and, optionally, other therapeutic and/or prophylactic ingredients. The carrier(s) must be “acceptable” in the sense of being compatible with the other ingredients of the formulation and not harmful to the recipient thereof.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound for use according to the invention, which matrices may be in form of shaped articles, e.g. films or microcapsules.

The compound for use according to the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The compound for use according to the present invention can be administered in a wide variety of oral and parenteral dosage forms. It will be obvious to those skilled in the art that the following dosage forms may comprise, as the active component, either a compound for use according to the invention or a pharmaceutically acceptable salt of a compound for use according to the invention.

For preparing pharmaceutical compositions from a compound for use according to the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the compound for use according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions exhibiting large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depends on the nature and severity of the disease being treated and the route of administration and is within the discretion of the physician, and it may be varied by titration of the dosage to the particular circumstances of this invention to produce the desired therapeutic effect. However, it is presently contemplated that pharmaceutical compositions containing of from about 0.01 to about 500 mg of active ingredient per individual dose, preferably of from about 0.1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.01 μg/kg i.v. and 0.1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method of treatment, prevention, prophylaxis or alleviation of a dyskinesia arising from the treatment of a subject suffering from Parkinson's disease with L-DOPA, which method comprises the step of administering to such a living animal body in need thereof, a therapeutically effective amount of Eltoprazine.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician in charge.

### BRIEF DESCRIPTION OF THE DRAWINGS

The present invention is further illustrated by reference to the accompanying drawing, in which:

FIG. 1 shows the effect, determined by integrated AIM score, of 5-HT_{1A} and 5-HT_{1B} agonists on L-DOPA-induced dyskinesia in partial DA lesioned rats, individually (8-OH-DPAT in Panel A, and CP-94253 in Panel B) and in combination (Panel C), and highlights the potent synergistic effect existing between the two agonists;

FIG. 2 shows the proposed mechanism of action of 5-HT_{1A} and 5-HT_{1B} agonists in the suppression of L-DOPA-induced dyskinesia;

FIG. 3 shows the effect of chronic administration of drugs that combine 5-HT_{1A} and 5-HT_{1B} activity. A long-lasting protection from the development of dyskinesia is provided in animals treated with the combination of 5-HT_{1A} and 5-HT_{1B} agonists from the very first dose of L-DOPA; and

FIG. 4 shows that Eltoprazine produced approximately 50% reduction of dyskinesia at 0.1 mg/kg compared to the control L-DOPA-only treated group, and a near-complete abolishment at 0.3 mg/kg dose.

### EXAMPLES

The invention is further illustrated with reference to the following examples, which are not intended to be in any way limiting to the scope of the invention as claimed.

### Example 1

### Effect of 5-HT_{1A} and 5-HT_{1B} Agonists Individually and in Combination on L-DOPA-Induced Dyskinesia in 6-OHDA-Lesioned Rats

In this experiment we determined the effect of 5-HT_{1A} and 5-HT_{1B} agonists on dyskinesia by applying the AIM (abnormal involuntary movements) scale developed by Lee and colleagues (see e.g. Lee C S, Cenci M A, Schulzer M, Bjorklund A: Embryonic ventral mesencephalic grafts improve levodopa-induced dyskinesia in a rat model of Parkinson's disease; Brain 2000 123 1365-1379) to evaluate the severity of L-DOPA-induced dyskinesia in rats.

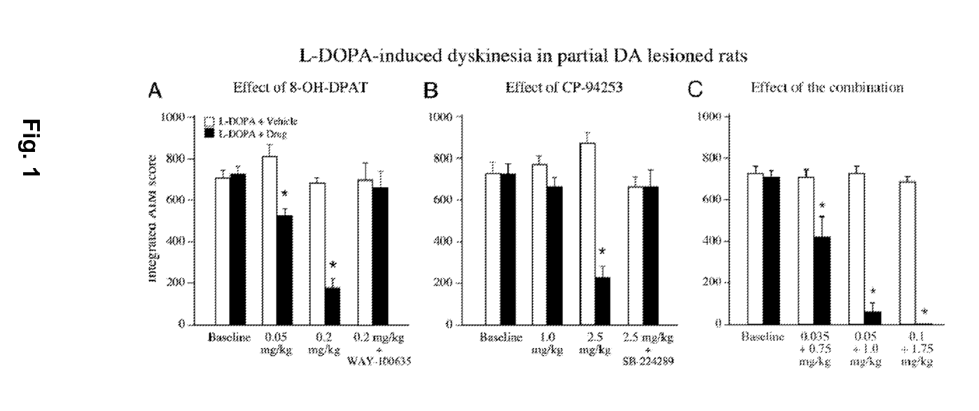

Two groups of L-DOPA primed, dyskinetic 6-OHDA-lesioned rat were challenged at each test with either L-DOPA-only, or L-DOPA plus the 5-HT_{1A} and 5-HT_{1B} agonists (8-OH-DPAT (8-hydroxy-2-(di-n-propylamino)tertraline), an agonist of the 5HT_{1A} and 5HT₇ receptors; WAY-100635 (N-(2-(4-(2-methoxyphenyl)-1-piperazinyl)ethyl)-N-(2-pyridinyl)cyclohexane carboxamide), a selective 5-HT_{1A} antagonist; CP-94253 (5-Propoxy-3-(1,2,3,6-tetrahydro-4-pyridinyl)-1H-pyrrolo[3,2-b]pyridine hydrochloride), a selective 5-HT_{1B} agonist; and SB-224289 (1′-Methyl-5-[[2′-methyl-4′-(5-methyl-1,2,4-oxadiazol-3-yl)biphenyl-4-yl]carbonyl]-2,3,6,7-tetrahydrospiro[furo [2,3-f]indole-3,4′-piperidine]oxalate), a selective 5-HT_{1B} receptor agonist), individually (FIG. 1, Panels A and B) or in combination (FIG. 1, Panel C).

As show in the figures, sub-threshold doses, which individually produced either marginal or no effect, in combination were able to dampen dyskinesia by more that 90% compared to the control L-DOPA-treated group, while higher dose completely suppressed dyskinesia.

### Example 2

### Pre-Synaptic Model of L-DOPA-Induced Dyskinesia

This example describes the proposed mechanism of action of 5-HT_{1A} and 5-HT_{1B} agonists in the suppression of L-DOPA-induced dyskinesia.

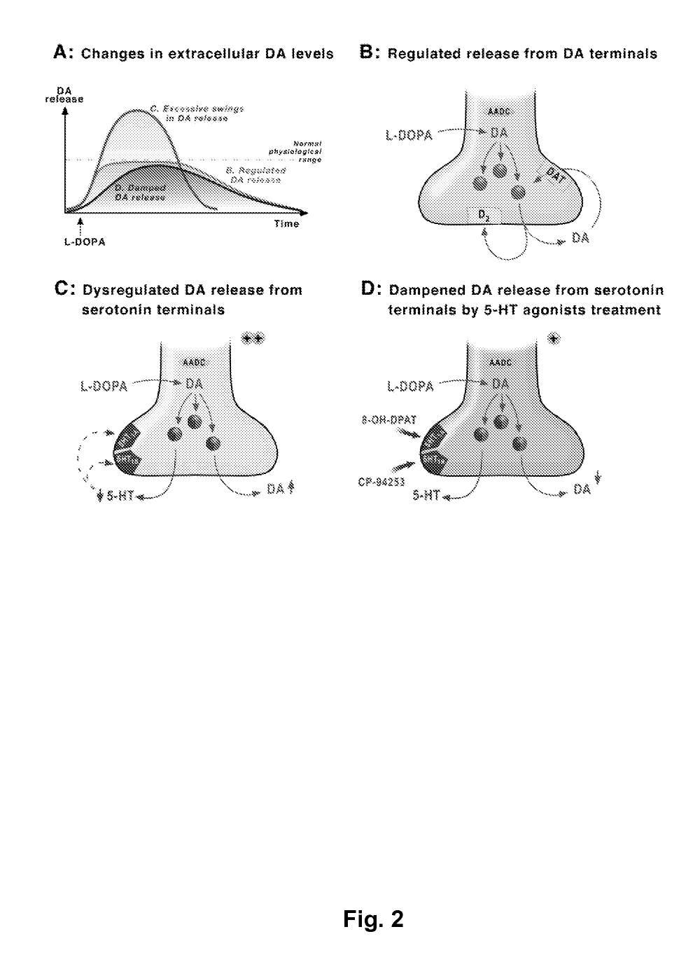

In early stages of PD the therapeutic effect of L-DOPA is sustained by the spared striatal dopamine terminals (FIG. 2, Panel B). At this stage of the disease, L-DOPA is taken up by the dopamine terminals, stored into vesicles and released in an activity-dependent manner.

Excessive swings in extracellular dopamine levels are prevented by auto-regulatory feed-back mechanism mediated by the dopamine transporter and the D₂ autoreceptors present on the dopaminergic terminals (FIG. 2, Panel A, green line B: Regulated DA release). As neurodegeneration progresses, fewer and fewer dopamine terminals will remain to mediate L-DOPA conversion and the serotonin terminals will come to play a major role (FIG. 2, Panel B). However, due to the lack of normal autoregulatory feed-back control and concomitant hyperactivation of serotonin terminals caused by depletion of endogenous serotonin by dopamine accumulating in the storage vesicles, dopamine released from serotonin terminals will be poorly regulated, resulting in uncontrolled, excessive swings in dopamine release (FIG. 2, Panel A, red line C: Excessive swings in DA release). The imbalance between the capacity of the serotonin terminals to release L-DOPA-derived dopamine, and the inability of the same neurons to provide a feed-back control mechanism to regulate the level of the neurotransmitter in the synaptic cleft, would be the driving force in the induction of dyskinesia.

According to this model, 5-HT_{1A} and 5-HT_{1B} agonists, particularly in combination, completely suppress L-DOPA-induced abnormal movements in 6-OHDA-lesioned rats by dampening synaptic dopamine levels to a more physiological range (FIG. 2, Panel C, and FIG. 2, Panel A, blue line D: Damped DA release). Note that for simplicity 5-HT_{1A} receptors are positioned at the terminal level, but are indeed located at the level of the cell body of serotonin neurons.

### Example 3

### Prevention of L-DOPA-Induced Dyskinesia in 6-OHDA-Lesioned Rats by Chronic Administration of 5-HT_{1A} and 5-HT_{1B} Agonists

This experiment demonstrates a further advantage of using drugs combining 5-HT_{1A} and 5-HT_{1B} activity, namely their capacity to induce long-lasting protection from the development of dyskinesia in animals treated with the combination of 5-HT_{1A} and 5-HT_{1B} agonists from the very first dose of L-DOPA.

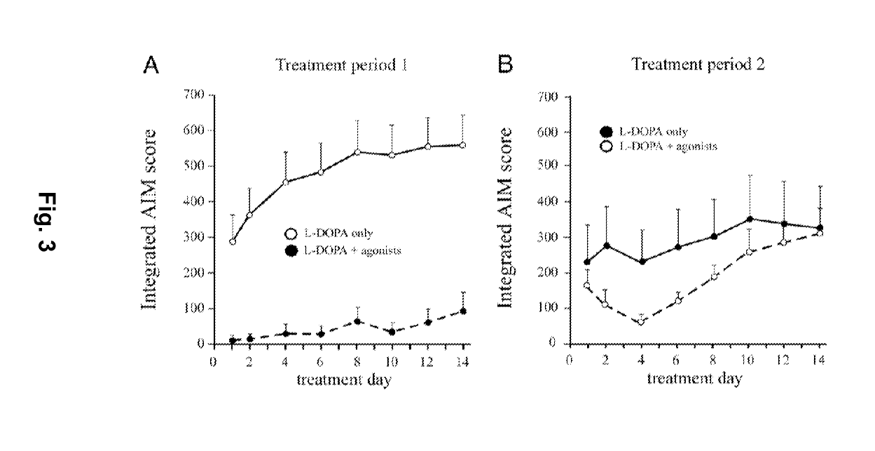

Two groups of 6-OHDA-lesioned rats were treated either with L-DOPA or L-DOPA plus a combination of 5-HT_{1A} and 5-HT_{1B} agonists (i.e. 8-OH-DPAT, 0.05 mg/kg; and CP-94253, 1.0 mg/kg s.c.) (treatment period 1). As shown in FIG. 3, Panel A, coadministration of the 5-HT agonists with L-DOPA resulted in a significant protection from the induction of dyskinesia compared to the L-DOPA-only treated group. Treatment were then switched so that animals receiving L-DOPA during treatment period 1 received L-DOPA plus combination of 8-OH-DPAT and CP-94253 during treatment period 2, while the animals treated with combination of 8-OH-DPAT and CP-94253 during treatment period 1 received L-DOPA-only during treatment period 2 (FIG. 3, Panel B).

Interestingly, the rats previously treated with the agonists seem to remain protected from a full development of dyskinesia even when agonists treatment is discontinue, since they never reached the dyskinesia score of the control group in the treatment period 1, and only few animals developed high dyskinesia (not shown).

### Example 4

### Antidyskinetic Effect of Eltoprazine in the Rat 6-OHDA Model

In this experiment we determined the antidyskinetic properties of Eltoprazine.

6-OHDA-lesioned rats were made dyskinetic and allocated into three well-balanced groups (n=6 per group), which were challenge on the day of the experiments with L-DOPA-only, L-DOPA plus Eltoprazine 0.1 mg/kg or L-DOPA plus Eltoprazine 0.3 mg/kg, respectively.

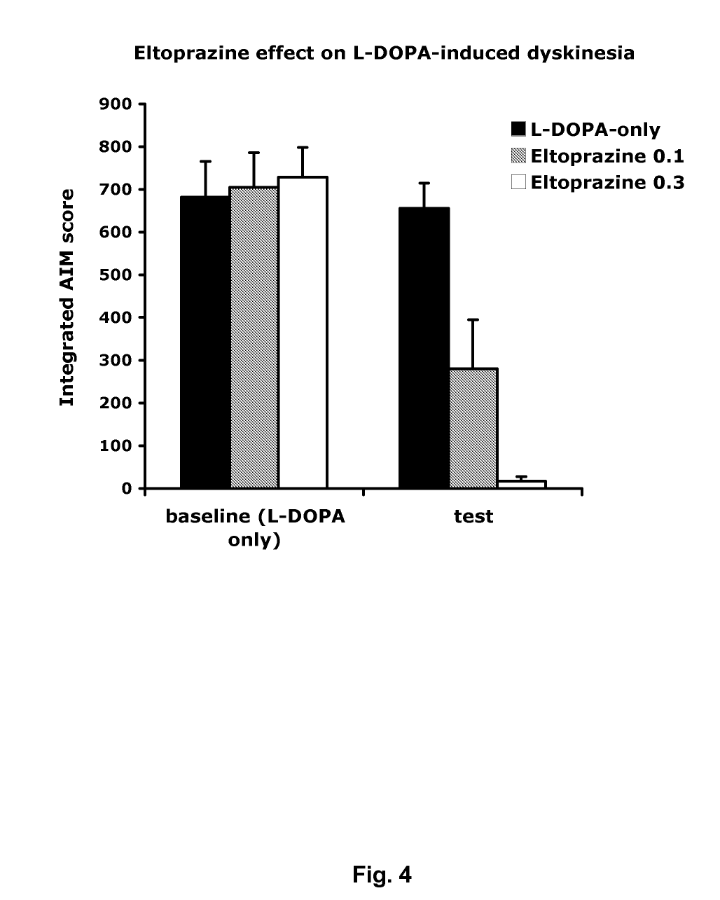

Limb, axial and orolingual dyskinesias were scored over the tow-hour test and the sum is reported here as the total score, and the results are presented in FIG. 4.

These data shows that Eltoprazine produced approximately 50% reduction of dyskinesia at 0.1 mg/kg compared to the control L-DOPA-only treated group, and a near-complete abolishment at 0.3 mg/kg dose, at which dose only few orolingual movements could be detected.

1. A compound (Eltoprazine) represented by Formula I, 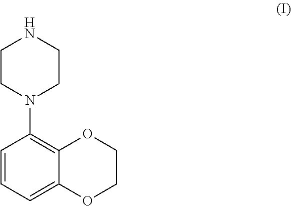
or a pharmaceutically acceptable addition salt thereof, or a prodrug thereof,
for use as a medicament for the treatment, prevention or alleviation of dyskinesias arising from the treatment of a subject suffering from Parkinson's disease with L-DOPA.

2. The use according to claim 1, wherein Eltoprazine hydrochloride is used.

3. Use of a compound (Eltoprazine) represented by Formula I, 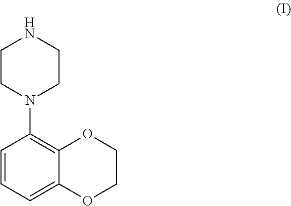
or a pharmaceutically acceptable addition salt thereof, or a prodrug thereof,
for the manufacture of a pharmaceutical composition/medicament for the treatment, prevention or alleviation of dyskinesias arising from the treatment of a subject suffering from Parkinson's disease with L-DOPA.

4. A kit of parts comprising at least two separate unit dosage forms (A) and (B):
(A) Eltoprazine; and
(B) L-DOPA; and optionally
(C) instructions for the simultaneous, sequential or separate administration of Eltoprazine of (A) and the L-DOPA of (B) to a patient in need thereof.

5. A method of treatment, prevention or alleviation of dyskinesias arising from the treatment of a subject suffering from Parkinson's disease with L-DOPA, which method comprises the step of administering to such a living animal body in need thereof, a therapeutically effective amount of a compound (Eltoprazine) represented by Formula I, 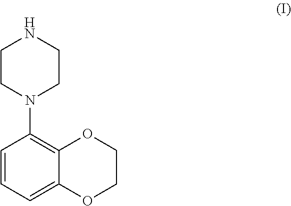
or a pharmaceutically acceptable addition salt thereof, or a prodrug thereof.

6. A method of treatment, prevention or alleviation of dyskinesias of a living animal body, including a human, suffering from Parkinson's disease, which method comprises the step of administering to such a living animal body in need thereof, a therapeutically effective amount of a combination of
(i) Eltoprazine; and
(ii) L-DOPA;
or pharmaceutically acceptable addition salts thereof.
